(19) United States
(12) Patent Application Publication
(10) Pub. No.: US 2023/0072133 A1
(43) Pub. Date: Mar. 9, 2023

(54) A BIFUNCTIONAL FUSION PROTEIN AND USES THEREOF

(71) Applicant: WUXI BIOLOGICS IRELAND LIMITED, Dundalk, Co Louth (IE)

(72) Inventors: Yunying CHEN, Shanghai (CN); Jing LI, Shanghai (CN); Jijie GU, Shanghai (CN)

(21) Appl. No.: 17/800,197

(22) PCT Filed: Feb. 24, 2021
(86) PCT No.: PCT/CN2021/077619

§ 371 Date: Aug. 16, 2022

(30) Foreign Application Priority Data
Feb. 25, 2020 (CN) PCT/CN2020/076658

(51) Int. Cl. C07K 16/28; C07K 14/71; A61P 35/00

(52) U.S. Cl. CPC C07K 16/2827; C07K 14/71; A61P 35/00; C07K 2319/00; A61K 38/00

(57) ABSTRACT

The present disclosure provides a fusion protein comprising at least a portion of TGFβRII and anti-PD-L1 antibody or antigen-binding portion thereof, methods of producing the fusion protein, methods of treating diseases or conditions using the fusion protein, and uses thereof.

### CROSS-REFERENCE TO RELATED APPLICATION

The application claims priority to International application PCT/CN2020/076658, filed on Feb. 25, 2020, the entire contents of which are hereby incorporated by reference.

### SEQUENCE LISTING

The present application is filed with a Sequence Listing in electronic form. The entire contents of the Sequence Listing are hereby incorporated by reference.

### FIELD

The present disclosure generally relates to bifunctional fusion proteins, a method for preparing the same and uses thereof.

### BACKGROUND

PD-1, one of the immune-checkpoint proteins, is an inhibitory member of CD28 family expressed on activated CD4+ T cells and CD8+ T cells as well as on B cells. Its ligand PD-L1 is a type 1 transmembrane protein that has been speculated to play a major role in suppressing the adaptive arm of immune system. The binding of PD-L1 to PD-1 transmits an inhibitory signal based on interaction with phosphatases (SHP-1 or SHP-2) via Immunoreceptor Tyrosine-Based Switch Motif (ITSM). As a result, this pathway inhibits T cell proliferation and T cell functions such as cytokine production and cytotoxic activity. PD-1/PD-L1 axis plays a major role in down-regulating the immune system [1, 2].

Monoclonal antibodies targeting PD-1 or PD-L1 can block PD-1/PD-L1 binding and boost the immune response against cancer cells. These drugs have shown a great deal of promise in treating certain cancers. Multiple approved therapeutic antibodies targeting PD-1/PD-L1 have been developed by several pharmaceutical companies, including Pembrolizumab (Keytruda), Nivolumab (Opdivo), Cemiplimab (Libtayo), Atezolizumab (Tecentriq), Avelumab (Bavencio) and Durvalumab (Imfinzi). These drugs have been shown to be effective in treating various types of cancer, including melanoma of the skin, non-small cell lung cancer, kidney cancer, bladder cancer, head and neck cancers, and Hodgkin lymphoma. They are also being studied for use against many other types of cancer [3].

The transforming growth factor-β (TGF-β) is a family of structurally related proteins that comprises of TGF-β, activins/inhibins, and bone morphogenic proteins (BMPs). Members of the TGF-β family control numerous cellular functions including proliferation, apoptosis, differentiation, epithelial-mesenchymal transition (EMT), and migration. TGF-β dysregulation has been implicated in carcinogenesis. In early stages of cancer, TGF-β exhibits tumor suppressive effects by inhibiting cell cycle progression and promoting apoptosis. However, in late stages TGF-β exerts tumor promoting effects, increasing tumor invasiveness, and metastasis. Furthermore, the TGF-β signaling pathway communicates with other signaling pathways in a synergistic or antagonistic manner and regulates cellular functions. Given the pivotal role of TGF-β in tumor progression, this pathway is an attractive target for cancer therapy [4]. Several therapeutic tools such as TGF-β antibodies, antisense oligonucleotides, and small molecules inhibitors of TGF-β receptor-1 (TGF-βR1) have shown immense potential to inhibit TGF-β signaling. Finally, in the interest of developing future therapies, further studies are warranted to identify novel points of convergence of TGF-β with other signaling pathways and oncogenic factors in the tumor microenvironment.

Recently, therapeutic agents simultaneously targeting the PD1/PD-L1 and TGF-β pathways, such as bifunctional protein containing TGFβRII extra-cellular domain (ECD) and anti-PD-L1 antibody, have been reported. However, the development of such fusion antibodies still has improvement space and clinical needs. In the present disclosure, a novel bifunctional protein comprising TGFβRII ECD and an anti-PD-L1 antibody is described. This fusion antibody protein has shown excellent protein stability and in vivo antitumor activity. These results demonstrate the great potential of this novel fusion antibody as a drug candidate for further preclinical studies.

### SUMMARY

These and other objectives are provided for by the present disclosure which, in a broad sense, is directed to compounds, methods, compositions and articles of manufacture that provide antibodies and proteins with improved efficacy. The benefits provided by the present disclosure are broadly applicable in the field of antibody therapeutics and diagnostics and may be used in conjunction with antibodies that react with a variety of targets.

In one aspect, the disclosure provides a fusion protein, comprising an antibody or antigen-binding portion thereof that specifically binds to PD-L1 fused with a human transforming growth factor β receptor (TGFβR) or a portion thereof capable of binding to TGFβ, wherein the antibody or antigen-binding portion thereof comprises:

a heavy chain CDR1 (HCDR1) comprising SEQ ID NO: 1 or an amino acid sequence that differs from SEQ ID NO: 1 by an amino acid addition, deletion and/or substitution of not more than 2 amino acids;

a HCDR2 comprising SEQ ID NO: 2 or an amino acid sequence that differs from SEQ ID NO: 2 by an amino acid addition, deletion and/or substitution of not more than 2 amino acids;

a HCDR3 comprising SEQ ID NO: 3 or an amino acid sequence that differs from SEQ ID NO: 3 by an amino acid addition, deletion and/or substitution of not more than 2 amino acids;

a light chain CDR1 (LCDR1) comprising SEQ ID NO: 4 or an amino acid sequence that differs from SEQ ID NO: 4 by an amino acid addition, deletion and/or substitution of not more than 2 amino acids;

a LCDR2 comprising SEQ ID NO: 5 or an amino acid sequence that differs from SEQ ID NO: 5 by an amino acid addition, deletion and/or substitution of not more than 2 amino acids; and

a LCDR3 comprising SEQ ID NO: 6 or an amino acid sequence that differs from SEQ ID NO: 6 by an amino acid addition, deletion and/or substitution of not more than 2 amino acids.

In some embodiments, the antibody or antigen-binding portion thereof as disclosed herein comprises:

a heavy chain CDR1 (HCDR1) comprising SEQ ID NO: 1; a HCDR2 comprising SEQ ID NO: 2; a HCDR3 comprising SEQ ID NO: 3; and

a light chain CDR1 (LCDR1) comprising SEQ ID NO: 4; a LCDR2 comprising SEQ ID NO: 5; and a LCDR3 comprising SEQ ID NO: 6.

In some embodiments, the antibody or antigen-binding portion thereof as disclosed herein comprises a heavy chain variable region (VH) and a light chain variable region (VL), wherein the VH comprises:

(A) an amino acid sequence as set forth in SEQ ID NO: 7;

(B) an amino acid sequence which is at least 85%, at least 90%, or at least 95% identical to SEQ ID NO: 7; or

(C) an amino acid sequence with addition, deletion and/or substitution of one or more (for example, 1, 2, 3, 4, 5, 6, 7, 8, 9 or 10) amino acids compared with SEQ ID NO: 7; and/or the VL comprises:

(A) an amino acid sequence as set forth in SEQ ID NO: 8;

(B) an amino acid sequence which is at least 85%, at least 90%, or at least 95% identical to SEQ ID NO: 8; or

(C) an amino acid sequence with addition, deletion and/or substitution of one or more (for example, 1, 2, 3, 4, 5, 6, 7, 8, 9 or 10) amino acids compared with SEQ ID NO: 8.

In some embodiments, the human TGFβR is selected from TGFβRII or TGFβRIII, preferably TGFβRII. In some preferred embodiments, rather than a full length TGFβRII, the fusion protein comprises a portion of human TGFβRII, which is the extra-cellular domain of TGFβRII.

In some embodiments, the human TGFβR or a portion thereof capable of binding to TGFβ as disclosed herein comprises:

(A) an amino acid sequence which is at least 85%, at least 90%, or at least 95% identical to the amino acid sequence of wild-type human TGFβRII;

(B) an amino acid sequence which is at least 85%, at least 90%, or at least 95% identical to the amino acid sequence of the extra-cellular domain of the wild-type human TGFβRII; or

(C) a portion of the wild-type human TGFβRII which retains at least 75%, at least 80%, at least 85%, at least 90%, or at least 95% of the binding capacity to TGFβ.

In some embodiments, the TGFβR or a portion thereof capable of binding to TGFβ comprises or consists of the amino acid sequence of the extra-cellular domain of the wild-type human TGFβRII, i.e. the amino acid sequence of SEQ ID NO: 9.

In some embodiments, the antibody or antigen-binding portion thereof comprised in the fusion protein is a full antibody, ScFv, Fab, F(ab′)2, or Fv fragment, such as a full antibody.

In some embodiments, the antibody or antigen-binding portion thereof comprises the VH region operably linked to an Fc region in the heavy chain. For example, the antibody or antigen-binding portion thereof may be a full antibody and comprises VH-CH1-hinge-Fc in the heavy chain, and VL-CL in the light chain.

In some embodiments, the antibody or antigen-binding portion thereof is IgG1, IgG2, IgG3 or IgG4 isotype, preferably IgG1 isotype.

In some embodiments, the Fc region of the antibody or antigen-binding portion thereof is operably linked to the N terminal of the human TGFβR or a portion thereof, optionally via a linker. The linker may be a peptide linker. In some embodiments, the linker comprises (G4S)n, with n=2-4.

In some embodiments, the antibody or antigen-binding portion thereof is a humanized or a fully human antibody, such as a fully human antibody. In some embodiments, the heavy chain and the light chain of the fusion protein comprises SEQ ID NOs: 10 and 11, respectively.

In one aspect, the disclosure provides an isolated nucleic acid molecule, comprising a nucleic acid sequence encoding the antibody or antigen-binding portion thereof and/or the human TGFβR or a portion thereof of the fusion protein as defined above.

In one aspect, the disclosure provides a vector comprising the nucleic acid molecule as defined herein. In one aspect, the disclosure provides a host cell comprising the nucleic acid molecule or the vector as defined herein.

In one aspect, the disclosure provides a pharmaceutical composition comprising the fusion protein as defined herein and a pharmaceutically acceptable carrier.

In one aspect, the disclosure provides a method for producing the fusion protein as defined herein, comprising the steps of:
expressing the fusion protein in the host cell as disclosed herein; and
isolating the fusion protein from the host cell.

In one aspect, the disclosure provides a method for modulating an immune response in a subject, comprising administering to the subject the fusion protein or the pharmaceutical composition as disclosed herein to the subject.

In one aspect, the disclosure provides a method for inhibiting growth of tumor cells associated with PD-1/PD-L1 in a subject, comprising administering an effective amount of the fusion protein or the pharmaceutical composition as disclosed herein to the subject.

In one aspect, the disclosure provides a method for preventing or treating cancer associated with PD-1/PD-L1 in a subject, comprising administering an effective amount of the fusion protein or the pharmaceutical composition as disclosed herein to the subject. Said cancer may be selected from colon cancer, lymphoma, lung cancer, liver cancer, cervical cancer, breast cancer, ovarian cancer, pancreatic cancer, melanoma, glioblastoma, prostate cancer, esophageal cancer, or gastric cancer. In some embodiments, the cancer to be prevented or treated is colon cancer or lung cancer, such as NSCLC.

In some embodiments, the fusion protein as disclosed herein is administered in combination with a chemotherapeutic agent, radiation and/or other agents for use in cancer immunotherapy.

In one aspect, the disclosure provides the fusion protein as disclosed herein for use in treating or preventing cancer associated with PD-1/PD-L1.

In one aspect, the disclosure provides use of the fusion protein as disclosed herein in the manufacture of a medicament for modulating an immune response or inhibiting growth of tumor cells associated with PD-1/PD-L1 in a subject.

In one aspect, the disclosure provides use of the fusion protein as disclosed herein in the manufacture of a medicament for treating or preventing cancer associated with PD-1/PD-L1.

In one aspect, the disclosure provides a kit for treating or diagnosing cancer, comprising a container comprising the fusion protein as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Further, the contents of all references, patents and published patent applications cited throughout this application are incorporated herein in entirety by reference.

### BRIEF DESCRIPTION OF FIGURES

FIG. 1 shows the result of antibodies binding to human TGF-β1, TGF-β2 and TGF-β3 when the TGF-βs are immobilized (A) or the antibodies are immobilized (B), determined by ELISA. Human IgG1 is an isotype control.

FIG. 2 shows the result of antibodies binding to human PD-L1 (A), cyno PD-L1 (B) and mouse PD-L1 (C) expressing cells determined by FACS.

FIG. 3 shows the antibodies simultaneously bind to PD-L1 and TGF-β1 when TGF-β1 (A) or human PD-L1 (B) were immobilized, determined by ELISA.

FIG. 4 shows the result of antibodies in blocking PD-1 binding to cell surface PD-L1 determined by FACS.

FIG. 5A shows the result of antibodies in blocking TGF-β1 signaling tested in RGA assay. FIG. 5B shows the result of antibodies in blocking human PD-1/PD-L1 signaling tested in RGA assay. Data were represented as Mean±SEM.

FIG. 6 shows the IL-2 (A) and IFN-γ (B) production results of antibodies in Allogeneic mixed lymphocyte reaction.

FIG. 7 shows the serum stability result of antibodies by dual binding ELISA test and PD-L1 binding FACS test.

FIG. 8 shows the mouse body weight change (A) and anti-tumor efficacy (B) of antibodies in HCC827 PBMC model in mice.

FIG. 9 shows the in vivo PK study result of WT1122 antibody.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a”, “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art. Moreover, any section headings used herein are for organizational purposes only and are not to be construed as limiting the subject matter described.

### Definitions

In order to better understand the disclosure, the definitions and explanations of the relevant terms are provided as follows.

The term “antibody” or “Ab” herein is used in the broadest sense, which encompasses various antibody structures, including polyclonal antibodies, monospecific and multispecific antibodies (e.g. bispecific antibodies). A native intact antibody generally is a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{H}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Chothia et al., (1989) Nature 342:878-883. Antibodies may be of different antibody isotypes, for example, IgG (e.g., IgG1, IgG2, IgG3 or IgG4 subtype), IgA1, IgA2, IgD, IgE or IgM antibody. In a broad sense, the fusion protein as disclosed herein comprising the anti-PD-L1 antibody or antigen-binding portion thereof also belongs to an antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. In certain embodiments, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

The term “variable domain” or “variable region” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

“Fc” with regard to an antibody refers to that portion of the antibody comprising the second (CH2) and third (CH3) constant regions of a first heavy chain bound to the second and third constant regions of a second heavy chain via disulfide bonding. The Fc region may also comprise part or whole of the hinge region. The Fc region of the antibody is responsible for various effector functions such as ADCC and CDC, but does not function in antigen binding. The capacity of antibodies to initiate and regulate effector functions through their Fc domain is a key component of their in vivo protective activity. Although the neutralizing activity of antibodies has been previously considered to be solely the outcome of Fab-antigen interactions, it has become apparent that their in vivo activity is highly dependent on interactions of the IgG Fc domain with its cognate receptors, Fcγ receptors (FcγRs), expressed on the surface of effector leukocytes.

The term “PD-L1” as used herein, when referring to the amino acid sequence of PD-L1 protein (Programmed death-ligand 1, e.g. as provided in NCBI GenBank ID: NP_054862.1), including full-length PD-L1 protein, or the extracellular domain of PD-L1 (PD-L1 ECD) or fragment containing PD-L1 ECD; Fusion protein of PD-L1 ECD, for example, fragment fused with IgG Fc from mice or human (mFc or hFc) is also included. Moreover, as understood by a person skilled in the art, PD-L1 protein would also include those into which mutations of amino acid sequence are naturally or artificially introduced (including but not limited to replacement, deletion and/or addition) without affecting the biological functions.

The term “an antibody that binds PD-L1” or an “anti-PD-L1 antibody” as used herein includes antibodies and antigen-binding fragments thereof that specifically recognize a PD-L1 protein, as well as antibodies and antigen-binding fragments thereof that specifically bind to a PD-L1 protein. As used herein, the expression “anti-PD-L1 antibody” includes both monovalent antibodies with a single specificity, as well as bispecific antibodies comprising a first antigen-binding site that binds PD-L1 and a second antigen-binding site that binds a second (target) antigen.

The term “TGFβ”, i.e. transforming growth factor beta (TGF-β), is a multifunctional cytokine belonging to the transforming growth factor superfamily that includes three different mammalian isoforms (TGF-β1 to 3, HGNC symbols TGFB1, TGFB2, TGFB3) and many other signaling proteins. TGFβ is involved in paracrine signalling and can be found in many different tissue types, including brain, heart, kidney, liver, bone, and testes. TGF-β dysregulation has been implicated in carcinogenesis. For example, it has been reported that there is a potential association between elevated PD-L1 expression and active TGF-β signaling in some human tumor samples (Justin M. David et al. Oncoimmunology. 2017; 6(10): e1349589).

The term “TGFβR”, i.e. TGFβ family receptors, can be grouped into three types, type I, type II, and type III. There are seven type I receptors, five type II receptors, and one type III receptor, for a total of 13 TGFβ superfamily receptors. “TGFβRII” or “TGFβ Receptor II”, as used herein, is meant a polypeptide having the wild-type human TGFβ Receptor Type 2 Isoform A sequence (e.g., the amino acid sequence of NCBI Reference Sequence (RefSeq) Accession No. NP-001020018), or a polypeptide having the wild-type human TGFβ Receptor Type 2 Isoform B sequence (e.g., the amino acid sequence of NCBI RefSeq Accession No. NP-003233) or having a sequence substantially identical to the wild-type amino acid sequences. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII may lack the signal sequence.

The term “monoclonal antibody” or “mAb”, as used herein, refers to a preparation of antibody molecules of single molecular composition. A monoclonal antibody displays a single binding specificity and affinity for a particular epitope.

The term “human antibody” or “fully human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “fusion protein”, as used herein, refers to a polypeptide having two (or more) portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. In certain embodiments, the two portions of the fusion protein are an antigen-binding portion thereof that specifically binds to PD-L1 and a human TGFβ receptor (TGFβR) or a portion thereof capable of binding to TGFβ, respectively. Such antibody comprising fusion proteins may also be viewed as antibodies (e.g. referred to as “fusion antibody”) in the present disclosure.

The terms “operably linked” refer to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The ability of “inhibit binding,” as used herein, refers to the ability of an antibody or a fusion protein to inhibit or block the binding of two molecules (e.g PD-1 and PD-L1, TGFβ1 and TGFβRII) to any detectable level. In certain embodiments, the binding of the two molecules can be inhibited at least 50% by the antibody or antigen-binding fragment thereof. In certain embodiments, such an inhibitory effect may be greater than 60%, greater than 70%, greater than 80%, or greater than 90%. In certain embodiments, the binding of PD-1 to cell surface PD-L1 can be blocked by the present fusion protein with an IC₅₀ of no more than 0.1 nM.

The term “epitope,” as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996).

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds to a PD-L1 protein is substantially free of antibodies that specifically bind antigens other than PD-L1 protein). An isolated antibody that specifically binds a human PD-L1 protein may, however, have cross-reactivity to other antigens, such as PD-L1 proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” as used herein, refers to the process by which nucleic acids are introduced into eukaryoticcells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human PD-L1 gene was transfected into 293F cells.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition.

Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For instance, the “an effective amount,” when used in connection with treatment of PD-1/PD-L1-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

Fusion Proteins Comprising an Anti-PD-L1 Antibody and TGFβR or Portions Thereof

The present application provides a fusion protein capable of specifically binding to both PD-L1 and TGF (e.g. TGFβ1 and TGFβ3), thus not only targets PD-L1 antigen or PD-L1 expressing cells, but also promotes local depletion of TGFβ in the microenvironment. In a broad sense, such fusion proteins can also be viewed as antibodies as they are capable of specifically binding to PD-L1, antagonizing PD-L1 activity and blocking the PD-1/PD-L1 signalling pathway. The fusion protein may also be referred to as “bifunctional protein” or “TGFβ trap-PD-L1 antibody fusion” herein.

The fusion protein herein may comprise (a) an antibody or antigen-binding portion thereof that specifically binds to PD-L1, and (b) human transforming growth factor β receptor (TGFβR) or a portion thereof capable of binding to TGFβ (also called a TGFβ trap), wherein (a) and (b) may be fused via a linker. The antibody or antigen-binding portion thereof may be of various formats, such as a full antibody, monospecific antibody, bispecific antibody, ScFv, domain antibody (SdAb), VHH, Fab, F(ab′)2 or Fv fragment and so on, as long as it has a specific binding affinity to PD-L1. The human transforming growth factor β receptor can be selected from TGFβRII and TGFβRIII, preferably TGFβRII. The fusion protein may comprise a portion of the wild-type TGFβRII that retains some or all of the binding capacity to TGFβ, for example, the fusion protein may comprise an extracellular domain (ECD) of TGFβRII.

The linker between (a) and (b) connects the C-terminus of the antibody or antigen-binding portion thereof that specifically binds to PD-L1 to the N-terminus of the human transforming growth factor β receptor (TGFβR) or a portion thereof capable of binding to TGFβ, or vice versa. In some embodiments, where the Fc region is absent, the linker may be connected to the C-terminus of the variable domain of the antibody (e.g. a Fab, domain Ab or a ScFv); alternatively, where the antibody is a full antibody or a heavy chain antibody, the linker may be connected to the C-terminus of the Fc region.

The combination of anti-PD-L1 and TGFβ trap in a single agent elicits a synergistic anti-tumor effect due to the simultaneous blockade of the interaction between PD-L1 on tumor cells and PD-1 on immune cells, and the neutralization of TGFβ in the tumor microenvironment. Compared with M7824, the fusion protein of the present disclosure exhibits better blockade of the PD-1/PD-L1 signalling pathway and more potently enhances IFNγ production, as demonstrated in the Examples.

Specifically, the present fusion protein provides one or more of the following properties:

(a) capable of binding to human PD-L1 with a K_{D} no more than 7×10⁻¹⁰ M and to TGFβ1 with a K_{D} no more than 2×10⁻¹² M, as determined by SPR;

(b) capable of binding to cyno PD-L1 with an EC₅₀ no more than 2 nM, as determined by FACS;

(c) capable of simultaneously binding to PD-L1 and TGFβ1;

(d) capable of PD-1/PD-L1 signaling blockade and TGF-β1 blockade;

(e) potently enhance IL-2 and IFNγ production in Allogeneic mixed lymphocyte reaction;

(f) being stable in accelerated stability study; and

(g) being stable in human serum for at least 14 days.

The Antibody or Antigen-Binding Portion Thereof that Specifically Binds to PD-L1

In some embodiments, the antibody or antigen-binding portion thereof that specifically binds to PD-L1 comprises one or more heavy chain CDRs (HCDRs) selected from at least one of the group consisting of:

(i) a HCDR1 comprising SEQ ID NO: 1 or a HCDR1 that differs in amino acid sequence from SEQ ID NO: 1 by an amino acid addition, deletion or substitution of not more than 2 amino acids;

(ii) a HCDR2 comprising SEQ ID NO: 2 or a HCDR2 that differs in amino acid sequence from SEQ ID NO: 2 by an amino acid addition, deletion or substitution of not more than 2 amino acids; and

(iii) a HCDR3 comprising SEQ ID NO: 3 or a HCDR3 that differs in amino acid sequence from SEQ ID NO: 3 by an amino acid addition, deletion or substitution of not more than 2 amino acids; and/or one or more light chain CDRs (LCDRs) selected from at least one of the group consisting of:

(i) a LCDR1 comprising SEQ ID NO: 4 or a LCDR1 that differs in amino acid sequence from SEQ ID NO: 4 by an amino acid addition, deletion or substitution of not more than 2 amino acids;

(ii) a LCDR2 comprising SEQ ID NO: 5 or a LCDR2 that differs in amino acid sequence from SEQ ID NO: 5 by an amino acid addition, deletion or substitution of not more than 2 amino acids; and

(iii) a LCDR3 comprising SEQ ID NO: 6 or a LCDR3 that differs in amino acid sequence from SEQ ID NO: 6 by an amino acid addition, deletion or substitution of not more than 2 amino acids.

In some embodiments, the antibody or antigen-binding portion thereof comprises a heavy chain variable region (VH) and a light chain variable region (VL), wherein the heavy chain variable domain comprises (i) a HCDR1 comprising or consisting of SEQ ID NO: 1; (ii) a HCDR2 comprising or consisting of SEQ ID NO: 2; and (iii) a HCDR3 comprising or consisting of SEQ ID NO: 3; and/or the light chain variable domain comprises: (i) a LCDR1 comprising or consisting of SEQ ID NO: 4; (ii) a LCDR2 comprising or consisting of SEQ ID NO: 5; and (iii) a LCDR3 comprising or consisting of SEQ ID NO: 6.

In some embodiments, the heavy chain variable domain comprises:

(i) the amino acid sequence of SEQ ID NO: 7; (ii) an amino acid sequence at least 85%, 90%, or 95% identical to SEQ ID NO: 7; or (iii) an amino acid sequence with addition, deletion and/or substitution of one or more amino acids compared with SEQ ID NO: 7; and/or

the light chain variable domain comprises:

(i) the amino acid sequence of SEQ ID NO: 8; (ii) an amino acid sequence at least 85%, at least 90%, or at least 95% identical to SEQ ID NO: 8; or (iii) an amino acid sequence with addition, deletion and/or substitution of one or more amino acids compared with SEQ ID NO: 8.

The fusion protein of the disclosure that comprises the above described antibody or antigen-binding portion thereof could bind to human PD-L1 with high affinity. The binding of an antibody of the disclosure to PD-L1 can be assessed using one or more techniques well established in the art, for instance, ELISA. The binding specificity of an antibody of the disclosure can also be determined by monitoring binding of the antibody to cells expressing a PD-L1 protein, e.g., flow cytometry. For example, an antibody can be tested by a flow cytometry assay in which the antibody is reacted with a cell line that expresses human PD-L1, such as HCC827 cells, or CHO-k1 or 293F cells that have been transfected to express PD-L1 on their cell surface. Additionally or alternatively, the binding of the antibody, including the binding kinetics (e.g., K_{D} value) can be tested in BIAcore binding assays. Still other suitable binding assays include ELISA or FACS assays, for example using a recombinant PD-L1 protein.

For instance, an antibody of the disclosure binds to a human PD-L1 protein with a K_{D} of 1×10⁻⁷ M or less, a K_{D} of 5×10⁻⁸ M or less, a K_{D} of 2×10⁻⁸ M or less, a K_{D} of 1×10⁻⁸ M or less, a K_{D} of 5×10⁻⁹ M or less, a K_{D} of 4×10⁻⁹ M or less, a K_{D} of 3×10⁻⁹ M or less, a K_{D} of 2×10⁻⁹ M or less, a K_{D} of 1×10⁻⁹ M or less, a K_{D} of 5×10⁻¹° M or less, or a K_{D} of 1×10⁻¹° M or less, as measured by Surface Plasmon Resonance. Alternatively, an antibody of the disclosure is capable to bind to a human or cyno PD-L1 expressing cell lines with an EC₅₀ of less than 5 nM, less than 4 nM, less than 3 nM, less than 2 nM, less than 1 nM or even less than 0.5 nM, as determined by FACS.

The assignment of amino acids to each CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted.

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the)(BLAST program (version 2.0) of Altschul, et al. (1990) J. MoI. Biol. 215:403-10. BLAST protein searches can be performed with the)(BLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs {e.g., XBLAST and NBLAST) can be used. See www.ncbi.nlm.nih.gov.

In other embodiments, the CDR amino acid sequences can be at least 90%, 91%, 92%, 93%, 94%, 95%, 96%, 97%, 98% or 99% identical to the respective sequences set forth above. In other embodiments, the amino acid sequences of the variable region can be at least 90%, 91%, 92%, 93%, 94%, 95%, 96%, 97%, 98% or 99% identical to the respective sequences set forth above.

Preferably, the CDRs of the isolated antibody or the antigen-binding portion thereof contain a conservative substitution of not more than 2 amino acids, or not more than 1 amino acid. The term “conservative substitution”, as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

The TGFβ Trap that Comprises Human Transforming Growth Factor βReceptor (TGF βR) or a Portion Thereof

TGFβ has three ligand isoforms, TGFβ1, 2 and 3, all of which exist as homodimers. There are also three TGFβ receptors (TGFβR), which are called TGFβR type I, II and III. TGFβRI is the signaling chain and cannot bind ligand. TGFβRII binds the ligand TGFβ1 and 3, but not TGFβ2, with high affinity. TGFβRIII is a positive regulator of TGFβ binding to its signaling receptors and binds all 3 TGFβ isoforms with high affinity. It has been reported that TGFβ1 and TGFβ2 has a predominant role in the tumor microenvironment and cardiac physiology, respectively, thus a therapeutic agent that neutralizes TGFβ1 but not TGFβ2 could provide an optimal therapeutic index by minimizing the cardiotoxicity without compromising the anti-tumor activity. Thus, TGFβRII is selected and the extracellular domain of TGFβRII has only 136 amino acid residues in length (SEQ ID No: 9), which makes it quite suitable for constructing an antibody-trap fusion protein.

In the present fusion protein, the human transforming growth factor β receptor (TGFβR) or a portion thereof may comprise:

(A) an amino acid sequence which is at least 85%, at least 90%, or at least 95% identical to the amino acid sequence of wild-type human TGFβRII;

(B) an amino acid sequence which is at least 85%, at least 90%, or at least 95% identical to the amino acid sequence of the extracellular domain of the wild-type human TGFβRII; or

(C) a portion of the wild-type human TGFβRII which retains at least 75%, at least 80%, at least 85%, at least 90%, or at least 95% of the binding capacity to TGFβ.

In some embodiments, the TGFβ trap comprised in the fusion protein herein is an extra-cellular domain of TGFβRII or a portion of the TGFβRII ECD. In some specific embodiments, the TGFβ trap comprises or consists of the sequence as set forth in SEQ ID NO: 9.

Nucleic Acid Molecules Encoding Fusion Protein of the Disclosure

In some aspects, the disclosure is directed to an isolated nucleic acid molecule, comprising a nucleic acid sequence encoding one of more of the following:

(i) the antibody or antigen-binding portion thereof, or the VH or VL domain of the antibody;

(ii) the human TGFβRII or a portion thereof of the fusion protein; and

(iii) the heavy chain or the light chain of the fusion protein.

In some aspects, the disclosure is directed to a vector comprising the nucleic acid sequence as disclosed herein. A vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, a PD-L1 antibody-encoding nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaP04-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the fusion protein in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison Wis.) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine synthetase as a selectable marker, such as the vectors described in Bebbington (1992) Biotechnology (NY) 10: 169-175.

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In an even further aspect, the disclosure relates to a host cell comprising the vector specified herein above.

Thus, the present disclosure also relates to a recombinant eukaryotic or prokaryotic host cell which produces a fusion protein of the present disclosure, such as a transfectoma. A fusion protein may be expressed in a recombinant eukaryotic or prokaryotic host cell, such as a transfectoma, which produces the fusion protein of the disclosure as defined herein.

Examples of host cells include yeast, bacterial, plant and mammalian cells, such as CHO, CHO-S, HEK, HEK293, HEK-293F, Expi293F, PER.C6 or NSO cells or lymphocytic cells. For example, in one embodiment, the host cell may comprise a first and second nucleic acid construct stably integrated into the cellular genome. In another embodiment, the present disclosure provides a cell comprising a non-integrated nucleic acid, such as a plasmid, cosmid, phagemid, or linear expression element, which comprises a first and second nucleic acid construct as specified above.

In a further aspect, the disclosure relates to a transgenic non-human animal or plant comprising nucleic acids encoding one or two sets of a heavy chain and light chain of the fusion protein as disclosed herein, wherein the animal or plant produces the fusion protein.

In a further aspect, the disclosure relates to a hybridoma which produces an antibody for use in a fusion protein as defined herein.

In one aspect, the disclosure relates to an expression vector comprising:

(i) a nucleic acid sequence encoding the the antibody or antigen-binding portion thereof, or the VH or VL domain of the antibody according to any one of the embodiments disclosed herein;

(ii) a nucleic acid sequence encoding the human TGFβRII or a portion thereof of the fusion protein according to any one of the embodiments disclosed herein;

(iii) a nucleic acid sequence encoding a heavy chain or a light chain of the fusion protein; or

(iv) the combinations of two or more of the above.

In one aspect, the disclosure relates to a nucleic acid construct encoding one or more amino acid sequences set out in the sequence listing.

In one aspect, the disclosure relates to a method for producing a fusion protein according to any one of the embodiments as disclosed herein, comprising the steps of culturing a host cell comprising an expression vector or more than one expression vectors as disclosed herein, expressing the fusion protein and purifying said fusion protein from the culture media. In one aspect, the disclosure relates to a host cell comprising an expression vector as defined above. In one embodiment, the host cell is a recombinant eukaryotic, recombinant prokaryotic, or recombinant microbial host cell.

Pharmaceutical Compositions

In some aspects, the disclosure is directed to a pharmaceutical composition comprising the fusion protein as disclosed herein and a pharmaceutically acceptable carrier.

Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the fusion protein as disclosed herein enhances the immune response against the antigen. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, in a solvent containing an antibody or an antigen-binding fragment of the present disclosure discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the fusion protein of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight per dose.

In any event, the fusion protein of the disclosure is preferably administered as needed to a subject in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the fusion protein of the instant disclosure will comprise multiple doses of the selected drug product over a period of weeks or months. More specifically, the fusion protein of the instant disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the fusion protein as disclosed herein in concentrations of from about 10 μg/ml to about 100 mg/ml. In certain selected embodiments, the concentrations of the fusion protein will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentrations of the fusion protein thereof will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

Applications of the Disclosure

In some aspects, the present disclosure provides a method of treating a disorder in a subject, which comprises administering to the subject (for example, a human) in need of treatment a therapeutically effective amount of the fusion protein as disclosed herein. For example, the disorder is a cancer.

A variety of cancers where PD-1/PD-L1 is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer. In some other embodiments, the cancer is lung cancer, such as NSCLC.

In some embodiments, examples of cancer include but not limited to B-cell cancers, including B-cell lymphoma (including low grade/follicular non-Hodgkin's lymphoma (NHL); small lymphocytic (SL) NHL; intermediate grade/follicular NEIL; intermediate grade diffuse NEIL; high grade immunoblastic NEIL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliierative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NEIL, front line low grade NEIL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NEIL, mantle cell lymphoma, follicle center lymphoma (follicular), intermediate grade diffuse NHL, diffuse large B-cell lymphoma, aggressive NHL (including aggressive front-line NHL and aggressive relapsed NEIL), NHL relapsing after or refractory to autologous stem cell transplantation, primary mediastinal large B-cell lymphoma, primary effusion lymphoma, high grade immunoblastic NHL, high grade lymphoblastic NEIL, high grade small non-cleaved cell NEIL, bulky disease NEIL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some other embodiments, the disorder is an autoimmune disease. Examples of autoimmune diseases that may be treated with the antibody or antigen-binding portion thereof include autoimmune encephalomyelitis, lupus erythematosus, and rheumatoid arthritis. The antibody or the antigen-binding portion thereof may also be used to treat or prevent infectious disease, inflammatory disease (such as allergic asthma) and chronic graft-versus-host disease.

Combined Use with Chemotherapies

The fusion protein as disclosed herein may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed fusion proteins prior to administration. More specifically, in certain embodiments selected anti-cancer agents will be linked to the unpaired cysteines of the antibodies to provide engineered conjugates. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic Rnases, such as extracellular pancreatic Rnases; Dnase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the fusion proteins of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3- dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the fusion protein with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed antibodies may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

Pharmaceutical Packs and Kits

Pharmaceutical packs and kits comprising one or more containers, comprising one or more doses of the fusion proteins are also provided. In certain embodiments, a unit dosage is provided wherein the unit dosage contains a predetermined amount of a composition comprising, for example, the fusion protein, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed composition is used for treating the neoplastic disease condition of choice.

The present disclosure also provides kits for producing single-dose or multi-dose administration units of antibodies or fusion proteins and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the fusion protein and, optionally, one or more anti-cancer agents in the same or different containers. The kits may also contain other pharmaceutically acceptable formulations, either for diagnosis or combined therapy. For example, in addition to the antibody or the antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the antibody and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above, the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

Sequence Listing Summary

Appended to the instant application is a sequence listing comprising a number of amino acid sequences. The following Table A provides a summary of the included sequences.

One illustrative fusion protein as disclosed herein is designated as WT1122-U14T1.G15-1.uIgG1 (abbreviated as WT1122).

TABLE A

| SEQ ID | |
|---|---|
| NO. | Description |
| 1 | Amino acid sequence of HCDR1 of the anti-PD-L1 antibody |
| 2 | Amino acid sequence of HCDR2 of the anti-PD-L1 antibody |
| 3 | Amino acid sequence of HCDR3 of the anti-PD-L1 antibody |
| 4 | Amino acid sequence of LCDR1 of the anti-PD-L1 antibody |
| 5 | Amino acid sequence of LCDR2 of the anti-PD-L1 antibody |
| 6 | Amino acid sequence of LCDR3 of the anti-PD-L1 antibody |
| 7 | Amino acid sequence of VH of the anti-PD-L1 antibody |
| 8 | Amino acid sequence of VL of the anti-PD-L1 antibody |
| 9 | Amino acid sequence of TGFβRII ECD domain |
| 10 | Amino acid sequence of the heavy chain of the fusion protein |
| 11 | Amino acid sequence of the light chain of the fusion protein |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Antigens, Benchmark Antibodies and Cell Lines

### 1.1 Preparation of Antigens

Human PD-L1 extracellular domain (ECD) antigen His tagged was purchased from Sino Biological (Cat #10084-H08H). Cynomolgus monkey (cyno) PD-L1 ECD antigen His tagged was purchased from Sino Biological (Cat #90251—CO8H). Human TGFβ1, TGFβ2 and TGF433 antigens were purchased from R&D Systems (Cat #7754-BH, 7754-BH/CF; Cat #302-B2, 302-B2/CF; Cat #8420-B3, 8420-B3/CF).

### 1.2 Establishment of PD-L1 Expressing Cell Lines

Human PD-L1 expressing cell line (W315-CHO-K1.hProl.C11), mouse PD-L1-expressing cell line (W315-293F.mPro1.C1) and cynomolgus monkey PD-L1-expressing cell line (W315-293F.cynoPro1.2A2) were generated as follows. Briefly, using Lipofectamine 2000 (ThermoFisher-11668027), CHO-K1 or 293F cells were transfected with the expression vector containing gene encoding full length human PD-L1 or cyno PD-L1 or mouse PD-L1. Cells were cultured in medium containing proper selection pressure. The stable cell lines were obtained by limited dilution.

### 1.3 Production of Benchmark Antibody (BMK)

TGFβRII ECD fusion anti-PD-L1 BMK antibody named as WT112-BMK2-IgG1 was constructed based on the sequence of M7824 in U.S. Pat. No. 9,676,863B2 from Merck Patent GmbH. The plasmid containing heavy chain gene and plasmid containing light chain gene were co-transfected to Expi293 cells with Expi293 expression kit (ThermoFisher-A14524). Cells were cultured for several days and supernatant was collected for protein purification.

### Example 2

### Generation of TGFβRII ECD Fused Anti-PD-L1 Antibodies

WT1122-U14T1.G15-1.uIgG1 is an anti-PD-L1 monoclonal antibody fused with TGFβRII ECD. The sequence of anti-PD-L1 antibody is from clone W3152-r11.135.5-zAb17-m6 in PCT/CN2020/110494 (incorporated herein by reference). The C-terminus of Fc was linked to the sequence of TGFβRII ECD, which was as the same as that in WT112-BMK2-IgG1 (SEQ ID NO: 9), via a linker. The linker between Fc and TGFβRII ECD is (G4S)4. The CDR sequences of WT1122-U14T1.G15-1.uIgG1 (abbreviated as “WT1122” herein) are listed in Table 1 below.

TABLE 1

| | | | |
|---|---|---|---|
| WT1122 | HCDR1 | HCDR2 | HCDR3 |
| | SEQ ID NO: 1 | SEQ ID NO: 2 | SEQ ID NO: 3 |
| | GFSLTENSVS | AVWSSGSTDYNSALKS | STYSNDFYYYFDY |
| | LCDR1 | LCDR2 | LCDR3 |
| | SEQ ID NO: 4 | SEQ ID NO: 5 | SEQ ID NO: 6 |
| | SGSELPKRYAY | KDSERPS | SSTYGDRKLPI |

TGFβRII ECD (SEQ ID NO: 9)
IPPHVQKSVNNDMIVTDNNGAVKFPQLCKFCDVRFSTCDNQKSCMSNC
SITSICEKPQEVCVAVWRKNDENITLETVCHDPKLPYHDFILEDAASPKC
IMKEKKKPGETFFMCSCSSDECNDNIIFSEEYNTSNPD

### Example 3

### In Vitro Characterization of WT1122

### 3.1 Human TGF-β Binding ELISA

The binding of antibodies to human TGF-β1, TGF-β2 and TGF-β3 was determined by ELISA. Plates were coated with human TGF-β1, TGF-β2 or TGF-β3 overnight at 4° C., respectively. After blocking and washing, various concentrations of testing antibodies were added to the plates and incubated at room temperature for 1 hour. The plates were then washed and incubated with HRP-labeled goat anti-human IgG antibody (Bethyl) for 1 hour. After washing, TMB substrate was added and the color reaction was stopped by 2M HCl. The absorbance at 450 nm and 540 nm was read using a microplate reader (SpectraMax M5e).

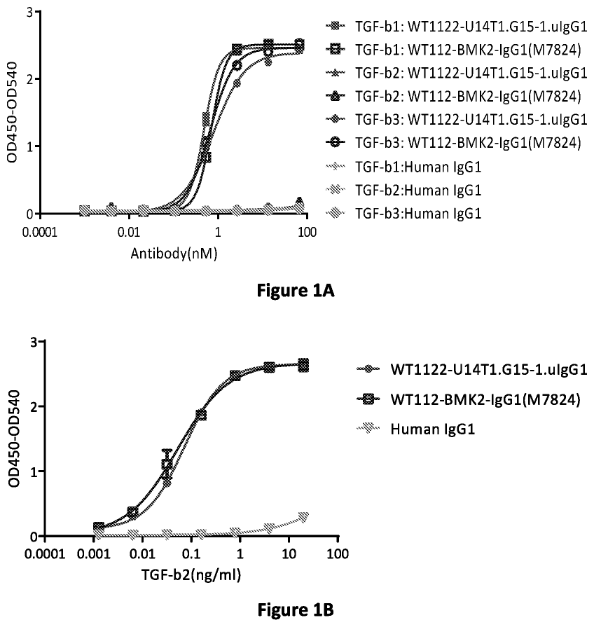

The binding curves of antibodies to plate coated human TGF-β1, TGF-β2 and TGF-β3 were shown in FIG. 1A. WT1122 shows similar affinity as WT112-BMK2-IgG1. They strongly bind to immobilized TGF-β1 (EC₅₀=0.5 nM) and TGF-β3 (EC₅₀=0.8 nM) but not to immobilized TGF-β2.

The binding of antibody to human TGF-β2 was also determined by ELISA immobilized with testing antibodies. After blocking and washing, various concentrations of TGF-β2 were added to the plates and incubated at room temperature for 1 hour. The plates were then washed and incubated with biotinylated TGF-β2 detection antibody (R&D, DY240) for 1 hour, followed by streptavidin-HRP for 1 hour. After washing, TMB substrate was added and the color reaction was stopped by 2M HCl. The absorbance at 450 nm and 540 nm was read using a microplate reader (SpectraMax M5e).

The binding curves of antibodies to soluble human TGF-β2 were shown in FIG. 1B. Immobilized WT1122 and WT112-BMK2-IgG1 can bind to soluble TGF-β2 with comparable EC₅₀ of 0.07 nM and 0.05 nM, respectively.

### 3.2 Human PD-L1 Binding FACS

Various concentrations of testing antibodies were incubated with hPD-L1 expressing W315-CHO-K1.hProl.C11 cells at 4° C. for 1 hour. After washing, the cells were incubated with PE-labeled goat anti-human IgG-Fc antibody (Jackson Immuno Research). Finally, the MFI of the cells was measured by a flow cytometer and analyzed by FlowJo.

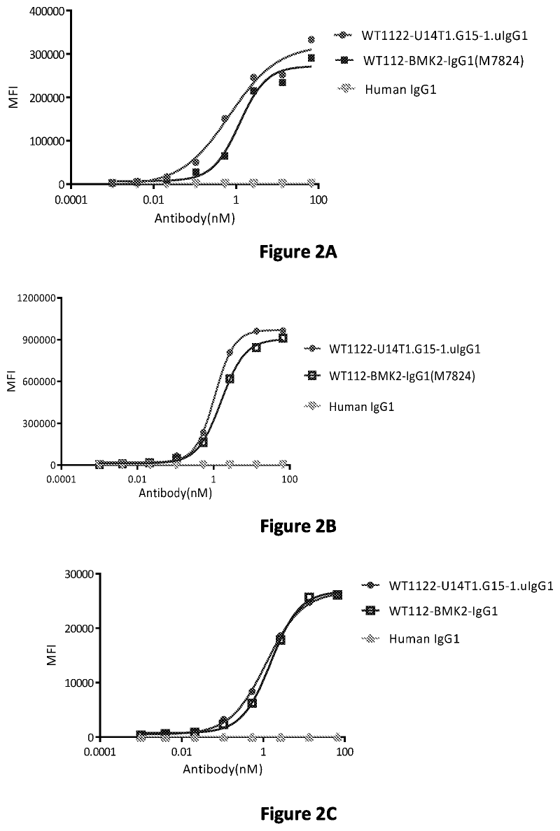

The binding curves to human PD-L1 transfected cells are shown in FIG. 2A. WT1122 and WT112-BMK2-IgG1 strongly bind to cell surface human PD-L1 with EC₅₀ of 0.7 nM and 1.21 nM, respectively.

### 3.3 Cross Species Binding FACS

The binding of testing antibodies to cynomolgus monkey or mouse PD-L1 was determined by FACS. Various concentrations of testing antibody were incubated with cynomolgus PD-L1-expressing W315-293F.cynoPro1.2A2 cells or mouse PD-L1 expressing W315-293F.mPro1.C1 cells at 4° C. for 1 hour, and then the binding of antibodies to the surface of the cells was detected by PE-labeled goat anti-human IgG-Fc antibody (Jackson Immuno Research). MFI of the cells was measured by a flow cytometer and analyzed by FlowJo.

The binding to cyno PD-L1 transfected cells was shown in FIG. 2B and to mouse PD-L1 trasfected cells in FIG. 2C. WT1122 and WT112-BMK2-IgG1 can strongly bind to cell surface cyno and mouse PD-L1. The cyno PD-L1 binding EC₅₀ of WT1122 and WT112-BMK2-IgG1 are 1.08 nM and 1.59 nM, respectively. The mouse PD-L1 binding EC₅₀ are 1.2 nM and 1.5 nM, respectively.

3.4 Simultaneous Binding with Human PD-L1 and Human TGF-β1

The simultaneous binding of testing antibodies to human TGF-β1 and human PD-L1 was determined by ELISA. Plates were coated with human TGF-β1 overnight at 4° C. After blocking and washing, various concentrations of testing antibodies were added to the plates and incubated at room temperature for 1 hour. The plates were then washed and incubated with biotinylated human PD-L1 ECD protein followed by streptavidin-HRP (Invitrogen) for 1 hour. After washing, TMB substrate was added and the color reaction was stopped by 2M HCl. The absorbance at 450 nm and 540 nm was read using a microplate reader (SpectraMax M5e).

Similarly, the simultaneous dual target binding was also tested by coating the plate with human PD-L1. After incubation with various concentrations of testing antibodies and then TGF-β1 antigen, the biotinylated human TGF-β1 detecting antibody (R&D, Cat 840117) and followed by streptavidin-HRP (Invitrogen) was added in the plate. Finally, TMB substrate was added and the color reaction was stopped by 2M HCl. The absorbance at 450 nm and 540 nm was read using a microplate reader (SpectraMax M5e).

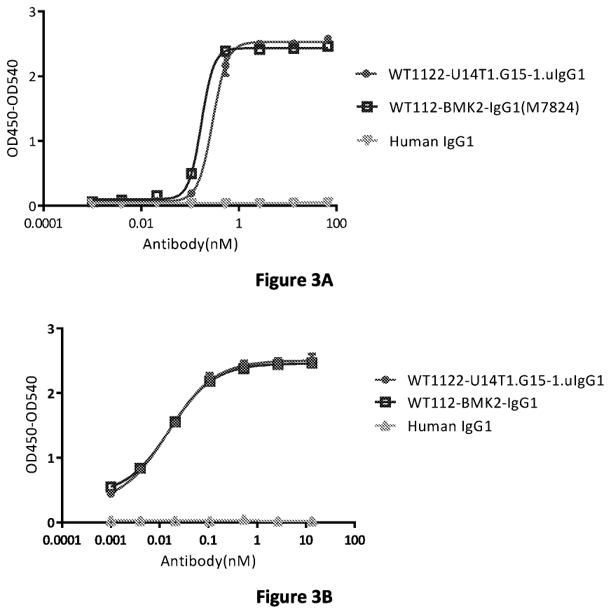

The result shown in FIG. 3A and FIG. 3B indicated that WT1122 and WT112-BMK2-IgG1 simultaneously bind to PD-L1 and TGF-β1 with EC₅₀ of 0.29 and 0.17 nM respectively when TGF-β1 was immobilized (FIG. 3A); EC₅₀ of 0.01 nM and 0.02 nM respectively when human PD-L1 was immobilized (FIG. 3B).

### 3.5 PD-1/PD-L1 Blockade by Competition FACS

Various concentrations of lead antibody, positive and negative control antibodies were mixed with mFc tagged human PD-1, and then incubated with human PD-L1 expressing transfected cells at 4° C. for 1 hour. The binding of human PD-1 to human PD-L1 expressing cells was detected by PE-labeled anti-mouse IgG Fc antibody (Abcam). MFI of the cells was measured by a flow cytometer and analyzed by FlowJo.

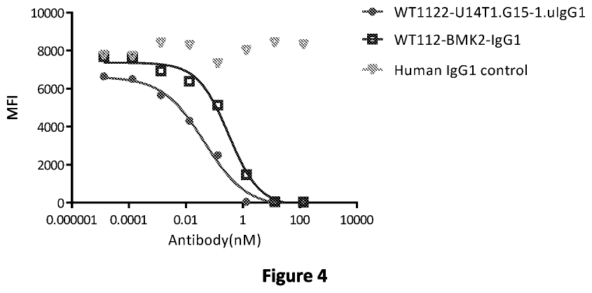

As shown in FIG. 4, WT1122 and WT112-BMK2-IgG1 block the binding of PD-1 to cell surface PD-L1 with IC₅₀ of 0.04 nM and 0.32 nM, respectively.

### 3.6 Reporter Gene Assay (RGA)

The blockade of TGF-β1 signaling was tested by a RGA assay. The RGA cell line was made by stably expressing full length of human Activin Receptor II B along with stably integrated SBE luciferase reporter gene. To test TGF-β1 signaling blockade activity of testing antibody, human TGF-β1 and various concentrations of antibodies were pre-mixed and added into the RGA cells and incubated overnight at 37° C., 5% CO₂. After incubation, reconstituted luciferase substrate (Promega, Cat E6130) was added and the luciferase intensity was measured by a microplate spectrophotometer.

The blockade of PD-1/PD-L1 signaling was tested by a RGA assay. PD-1 RGA cell line was made by stably expressing full length of PD-1 along with NFAT luciferase reporter gene in Jurkat E6-1 cells. The PD-1 RGA cells were incubated with human PD-L1 expressing artificial APC (a human PD-L1 and OKT3 sc-Fv expressing CHO-K1 cell) in the presence of various concentrations of testing antibodies for 4-6 hours at 37° C., 5% CO₂. After incubation, reconstituted luciferase substrate was added and the luciferase intensity was measured by a microplate spectrophotometer.

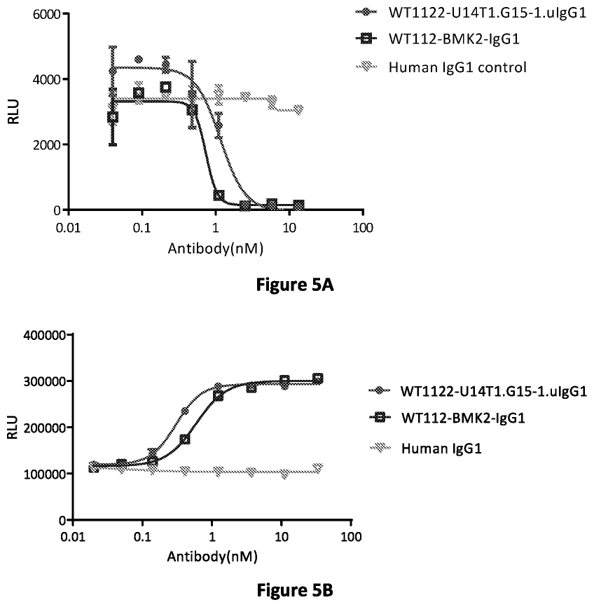

As shown in FIG. 5A, WT1122 displays comparable TGF-β1 blockade IC₅₀ of 1.2 nM to WT112-BMK2 (IC₅₀=0.7 nM). As shown in FIG. 5B, WT1122 and WT112-BMK2-IgG1 show strong hPD-1/PD-L1 signaling blockade activity in the RGA assay. The IC₅₀ of WT1122 and WT112-BMK2-IgG1 are 0.31 nM and 0.59 nM, respectively.

### 3.7 Allogeneic Mixed Lymphocyte Reaction (Allo-MLR)

Human peripheral blood mononuclear cells (PBMCs) were freshly isolated from healthy donors using Ficoll-Paque PLUS (Stem Cell) gradient centrifugation. Monocytes were isolated using CD14 MicroBeads (Miltenyi Biotec) according to the manufacturer's instructions. Cells were cultured in medium containing GM-CSF (Amoytop Biotech) and IL-4 (R&D) for 5 to 7 days to generate dendritic cells (DC). Human CD4⁺ T cells were isolated using human CD4⁺ T cell enrichment kit (Stem Cell) according to the manufacturer's protocol. Purified CD4⁺ T cells were co-cultured with allogeneic immature DCs (iDCs) in the presence of various lead antibody, positive and negative control antibodies in 96-well plates. The plates were incubated at 37° C., 5% CO₂. Supernatants were harvested for IL-2 and IFN-γ test at day 3 and day 5, respectively.

Human IL-2 and IFN-γ release were measured by ELISA using matched antibody pairs. Recombinant human IL-2 (R&D) and IFN-γ (PeproTech) were used as standards, respectively. The plates were pre-coated with capture antibody specific for human IL-2 (R&D) or IFN-γ (Pierce), respectively. After blocking, 50 μL of standards or samples were pipetted into each well and incubated for 2 hours at ambient temperature. Following removal of the unbound substances, the biotin-conjugated detecting antibody specific for corresponding cytokine was added to the wells and incubated for one hour. HRP-labeled streptavidin was then added to the wells for 30 minutes incubation at ambient temperature. The color was developed by dispensing 50 μL of TMB substrate, and then stopped by 50 μL of 2N HCl. The absorbance was read at 450 nm and 540 nm using a microplate spectrophotometer.

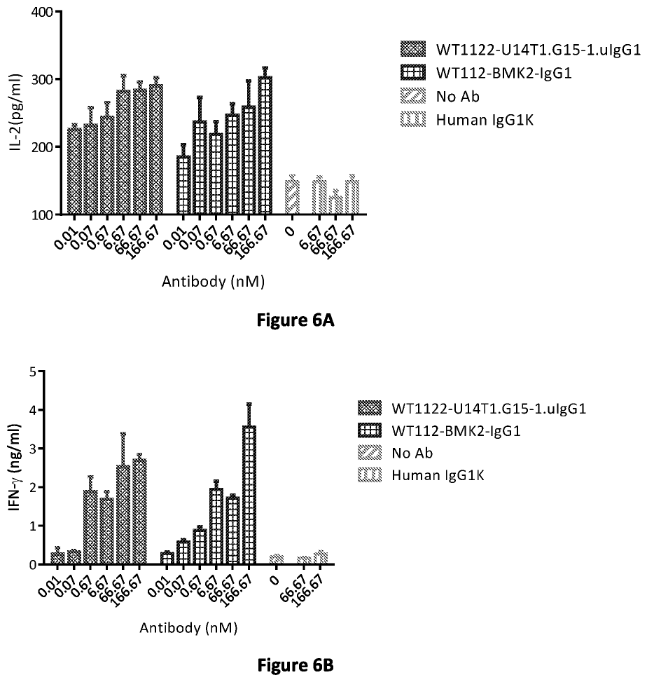

The results shown in FIG. 6A-6B demonstrate that WT1122 and WT112-BMK2-IgG1 can enhance the IL-2 production (FIG. 6A) and IFNγ production (FIG. 6B) in human CD4⁺ T cells allo-MLR assay in a dose dependent manner.

### 3.8 Serum Stability

WT1122 was incubated in freshly isolated human serum (serum content >95%) at 37° C. in a 5% CO₂ incubator. At indicated time point, aliquot of serum treated samples were removed from the incubator and snap frozen in liquid N₂, and then stored at −20° C. until ready for test. The samples were quickly thawed immediately prior to the stability test. The procedure of simultaneous binding ELISA, human TGF-β1 binding ELISA and human PD-L1 binding FACS were described above.

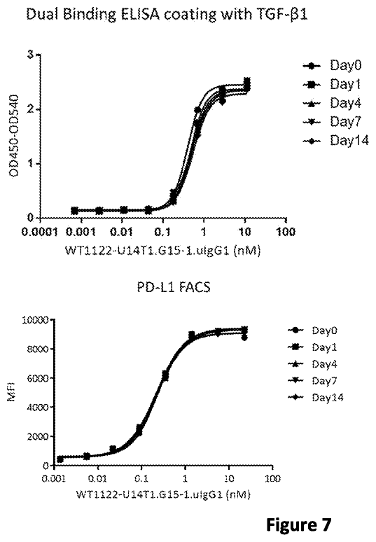

As shown in FIG. 7, these WT1122 samples show normal binding to targets, suggesting that the antibody is stable in human serum for at least 14 days.

### 3.9 Antibody Protein Accelerated Stability Study

### 3.9.1 Sample Treatment and Accelerated Stability Study

WT1122 was dialyzed via dialysis bag (Spectrum-888-10987, MWCO 3.5 kDa) into PBS buffer and then diluted to 2 ug/ml. An accelerated stability study was conducted by incubation of testing antibody at 4° C., 25° C. and 40° C. respectively for 1 day, 4 days and 7 days, as well as freeze-thawed for 3 cycles at −80° C. (Table 2). After incubation at each testing condition, the sample visual inspection was performed immediately to carefully detect the presence of any particulates. All the samples appeared as clear solution free of particulates. The antibody stability of each treated sample was analyzed by SDS-PAGE, analytic SEC-HPLC, DSF and DLS assays. The results shown in Table 2 indicated the WT1122 is stable in the accelerated stability study.

### 3.9.2 Thermo Stability by DSF

A DSF assay was performed using Real-Time Fluorescent Quantitative PCR (QuantStudio 7 Flex, Thermo Fisher Scientific). Briefly, 19 μL of antibody solution was mixed with 1 μL of 62.5×SYPRO Orange solution (Invitrogen) and added to a 96 well plate (Biosystems). The plate was heated from 26° C. to 95° C. at a rate of 0.9° C./min, and the resulting fluorescence data were collected. The negative derivatives of the fluorescence changes with respect to different temperatures were calculated, and the maximal value was defined as melting temperature. Data collection and Tm calculation was conducted automatically by operation software (QuantStudio™ Real-Time PCR Software v1.3). The Tm of WT1122 in PBS buffer is about 65.6° C. (Table 2).

### 3.9.3 Molecule Radius Measurement by DLS

Molecule radius measurement was investigated using DynaPro Plate Reader III dynamic light scattering (DLS) instrument (Wyatt Dynapro™). Five acquisitions were collected for each protein sample and each acquisition time was 5 s. Each well contained 7.5 μL of solution in 1536 plate (Aurora microplate). For each measurement, the diffusion coefficient was determined. Radius were calculated automatically by the operation software (DYNAMICS 7.8.1.3). The results shown in table 2 suggested that the radius ranges of samples after different treatments are from 13.6 nm to 14.9 nm, which are comparable to the sample freshly thawed from −80° C. (Radius of TO is 13.7 nm).

TABLE 2
Accelerated stability result in PBS

| | | | DLS | | DSF |
|---|---|---|---|---|---|
| | | Conc. | Radius | SEC (HMW/ | Tm |
| Treatment | Appearance | (mg/mL) | (nm) | Mono/LMW % | ° C. |
| T0 | particle-free | 2.05 | 13.7 | 3.05/96.95 | 65.6 |
| 3X | particle-free | 2.02 | 14.8 | 3.83/96.17 | 65.9 |
| 4° C.-1 | particle-free | 2.02 | 14.8 | 3.22/96.78 | 65.7 |
| Day | | | | | |
| 4° C.-4 | particle-free | 1.98 | 13.7 | 3.19/96.81 | 65.7 |
| Days | | | | | |
| 4° C.-7 | particle-free | 2.03 | 13.6 | 3.02/96.98 | 65.7 |
| Days | | | | | |
| 25° C.-1 | particle-free | 2.02 | 14.3 | 3.11/96.89 | 65.9 |
| Day | | | | | |
| 25° C.-4 | particle-free | 2.05 | 14.6 | 3.12/96.69/0.19 | 65.7 |
| Days | | | | | |
| 25° C.-7 | particle-free | 2.03 | 14.1 | 2.95/96.81/0.24 | 65.9 |
| Days | | | | | |
| 40° C.-1 | particle-free | 2.05 | 14.9 | 3.1/96.64/0.26 | 65.7 |
| Day | | | | | |
| 40° C.-4 | particle-free | 2.03 | 14.3 | 3.01/96.47/0.53 | 65.9 |
| Days | | | | | |
| 40° C.-7 | particle-free | 2.03 | 14.9 | 3.22/95.96/0.82 | 65.9 |
| Days | | | | | |

T0: It has been frozen and thawed once (from −80° C.).
3X: The sample was frozen and thawed for 3 more times than T0.

### 3.10 Full Kinetic Binding Affinity to PD-L1 by SPR

WT1122 binding affinity to human, mouse and cyno PD-L1 was detected by SPR assay using Biacore 8K. Antibody was captured on an anti-human IgG Fc antibody immobilized CM5 sensor chip (GE). Human or cyno PD-L1 at different concentrations were injected over the sensor chip at a flow rate of 30 μL/min for an association phase of 180 s, followed by 3600 s dissociation. Mouse PD-L1 at different concentrations were injected over the sensor chip at a flow rate of 30 μL/min for an association phase of 120 s, followed by 1200 s dissociation. The chip was regenerated by 10 mM glycine (pH 1.5) after each binding cycle.

The sensorgrams of blank surface and buffer channel were subtracted from the test sensorgrams. For WT1122 binding to mouse PD-L1, 0-300 s curves were used in the fitting process. The experimental data was fitted by 1:1 model using Langmiur analysis. Molecular weight of 40 kDa was used to calculate the molar concentration of human, mouse and cyno PD-L1. As shown in Table 3, WT1122 has similar affinity to human and cyno PD-L1.

TABLE 3
Binding affinity of WT1122 to human, cyno and mouse PD-L1

| Analyte | Ligand | ka (l/Ms) | kd (1/s) | KD (M) |
|---|---|---|---|---|
| Human PD-L1 | WT1122 | 2.41E+06 | 1.68E−03 | 6.95E−10 |
| Cyno PD-L1 | | 3.03E+06 | 1.35E−03 | 4.46E−10 |
| Mouse PD-L1 | | / | / | 1.08E−06 |

### 3.11 Full Kinetic Binding Affinity to TGFβ by SPR

Antibody binding affinity to TGFβ was detected by SPR assay using Biacore 8K. Each antibody was immobilized on CM5 sensor chip (GE). Human TGFβ1 and TGFβ3 at different concentrations were injected over the sensor chip at a flow rate of 50 uL/min for an association phase of 240 s, followed by 1200 s dissociation. Human TGFβ2 at different concentrations were injected over the sensor chip at a flow rate of 50 uL/min for an association phase of 240 s, followed by 300 s dissociation. The chip was regenerated by 10 mM glycine (pH 1.5) after each binding cycle.

The sensorgrams of blank surface and buffer channel were subtracted from the test sensorgrams. The experimental data was fitted by 1:1 model using Langmiur analysis. Molecular weight of 22 kDa, 24 kDa and 22 kDa were used to calculate the molar concentration of analyte TGFβ1, TGFβ2 and TGFβ3, respectively. The results are shown in

TABLE 4
Binding affinity of WT1122 to TGFβ by SPR.

| Analyte | Ligand | kₐ (1/Ms) | k_{d} (1/s) | KD (M) |
|---|---|---|---|---|
| TGF-β1 | WT1122 | 1.25E+08 | 2.06E−04 | 1.65E−12 |
| TGF-β2 | | 4.02E+07 | 1.49E−02 | 3.69E−10 |
| TGF-β3 | | 5.78E+07 | 9.35E−05 | 1.62E−12 |

### 3.12 Full Kinetic Binding Affinity to FcRn by SPR

Antibody binding affinity to human FcRn (ARCO, FCM-H5286) was detected using Biacore 8K. Each antibody was immobilized on CM5 sensor chip (GE). Human FcRn at different concentrations were injected over the sensor chip at a flow rate of 30 uL/min for an association phase of 60 s, followed by 90 s dissociation. The chip was then regenerated by 10 mM glycine (pH 1.5) after each binding cycle. The sensorgrams of blank surface and buffer channel were subtracted from the test sensorgrams. The experimental data was fitted by steady-state affinity model. Molecular weight of 45 KDa was used to calculate the molar concentration of analyte FcRn. The running buffer was PBST, pH 6.0.

The affinities of WT1122 and WT112-BMK2-IgG1 to FcRn are similar (Table 5).

TABLE 5
Affinity to FcRn by SPR

| Analyte | Ligand | K_{D} (M) |
|---|---|---|
| Human FcRn | WT1122 | 2.57E−06 |
| | WT112-BMK2-IgG1 | 1.89E−06 |

### Example 4

### In Vivo Anti-Tumor Efficacy Study

### 4.1 In Vivo Anti-Tumor Efficacy Study in HCC827 PBMC Model

WT1122 anti-tumor efficacy study was tested in HCC827 model in NCG female mice. Female NCG mice (Nanjing Galaxy Biopharmaceutical Co. Ltd) of 13-14 week-old were used in the study. HCC827 cells were maintained in vitro as a monolayer culture in RPMI 1640 medium supplemented with 10% fetal bovine serum, 100 U/mL penicillin and 100 μg/mL streptomycin at 37° C. in an atmosphere of 5% CO₂ in air. The tumor cells were routinely sub-cultured twice a week with 0.25% trypsin-EDTA treatment. The cells growing in an exponential growth phase were harvested and counted for tumor inoculation.

For the therapeutic model, each mouse was inoculated subcutaneously at the right front flank with HCC827 tumor cells (5.0×10⁶ cells with 50% of matric gel in 200 μL PBS). When the average tumor volume reached approximately to 173 mm³, animals were randomly grouped into 5 groups and each group contained 7 mice. The mice received human PBMC (5.0×10⁶, Hemocare, Lot No. 19057819) by intravenous injection; 1-2 h post PBMC implantation, animals were treated with drug by injections intraperitoneally at day0, day3, day7 and day10 for total 4 injections. 4 groups (G2-G5) were injected with 20 mg/kg of WT112-BMK2-IgG1 and WT1122 at 0.2 mg/kg, 2 mg/kg and 20 mg/kg, respectively. The control group (G1) received injections with vehicle-PBS. The day of the first injection was considered as day 0. For all tumor studies, mice were weighed and tumor growth was measured twice a week using calipers.

All the procedures related to animal handling, care and the treatment in the study were performed according to the guidelines approved by the Institutional Animal Care and Use Committee (IACUC) of Shanghai SIPPR-BK Laboratory Animal Co., Ltd and following the guidance of the Association for Assessment and Accreditation of Laboratory Animal Care (AAALAC). Tumor volume was calculated with the formula (½ (length×width²). The results were represented by mean and the standard error (Mean±SEM). Data were analyzed using Two way ANOVA Tukey's multiple comparisons test with Graphpad Prism 6.0 and p<0.05 was considered to be statistically significant.

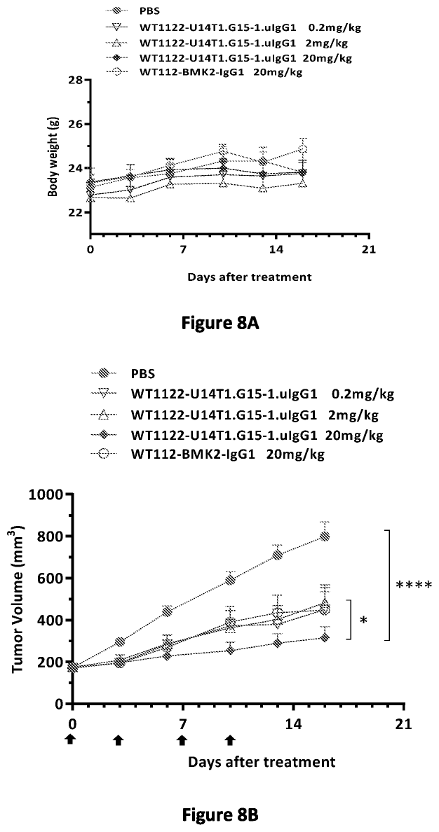

As shown in FIG. 8A, all mice were normal during the experiment without obvious bodyweight loss, suggesting that antibodies were not toxic. As shown in FIG. 8B, at the 16 days after the first dosing, the mean tumor volume of vehicle group was 798 mm³, which indicated HCC827 model was well established. Compared with vehicle group, WT1122 showed potent antitumor effect and significantly inhibited tumor growth. The TGI at day 16 of each group was 55.91% for WT112-BMK2-IgG1 at 20 mg/kg, 55.42%, 50.72% and 77.13% for WT1122 at 0.2 mg/kg, 2 mg/kg and 20 mg/kg, respectively. WT1122 exhibited superior antitumor activity to BMK2 at high dose (p<0.05), comparable antitumor activity at low and mid doses (p>0.05).

4.2 In Vivo PK Study of WT1122 at 30 mg/kg in Cyno Monkey

Two adult cyno monkey animals (one male+one female) were injected with 30 mg/Kg WT1122 via intravenous injection. Bodyweight, food consumption and clinical observation was done daily. Electrocardiograph (ECG), blood and serum samples were collected at different time points. Blood was collected into tubes containing EDTA-K2 for hematology and 2.0 mL blood was collected into tubes without additive for serum chemistry determination. Standard clinical chemistry and hematological analysis were performed. For PK and immunology analysis, approximately 1.2 mL blood was collected, about 0.5 mL serum was harvested by centrifugation at 3,500 rpm and 4° C. for 15 minutes, and stored frozen at approximately −70° C. or lower. Daily clinical observation was performed and recorded, All the procedures related to animal handling, care and the treatment in the study were performed according to the guidelines approved by the Institutional Animal Care and Use Committee (IACUC) of Guangzhou University of Chinese Medicine Science and Technology Park Co., Ltd. following the guidance of the Association for Assessment and Accreditation of Laboratory Animal Care (AAALAC). The concentrations of WT1122 in serum were determined by using a bioanalytical ELISA method. The serum concentration was subjected to a non-compartmental pharmacokinetic analysis by using the Phoenix WinNonlin software (version 8.1, Pharsight, Mountain View, Calif.). The linear/log trapezoidal rule was applied in obtaining the PK parameters, data was expressed with Mean±SD

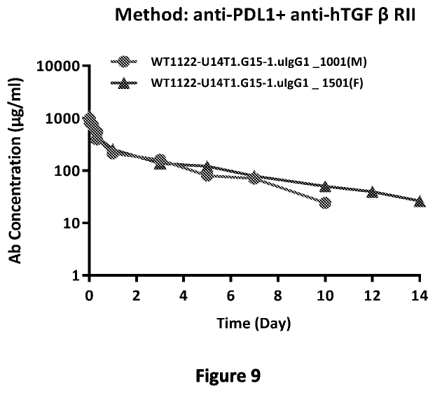

Animals tolerated well to single intravenous dose of WT1122 at 30 mg/kg, no obvious side-effect was observed, including ECG, daily clinic observation, bodyweight and major hematologic parameters (ALT, AST, WBC, RBC, PLT, QTc etc.). As shown in table 6 and FIG. 9, terminal half-life was 92.1 h, AUC_{0-inf} was 34993 h*μg/mL, clearance was 20.9 ml/day/kg.

TABLE 5
PK profile of WT1122 after single
intravenous injection at 30 mg/kg

| | Male | Female | | |
|---|---|---|---|---|
| PK Parameters | (1001) | (1501) | Mean | SD |
| Rsq_adj | 0.908 | 0.983 | — | — |
| No. points used for T1/2 | 4.00 | 7.00 | — | — |
| Cₘₐₓ (μg/mL) | 986 | 1140 | 1063 | 109 |
| T1/2 (h) | 82.6 | 102 | 92.1 | 13.5 |
| Cl (mL/day/kg) | 23.6 | 18.2 | 20.9 | 3.78 |
| Vdss (mL/kg) | 70.6 | 79.0 | 74.8 | 5.96 |
| MRT₀₋ₗₐₛₜ (h) | 71.8 | 103 | 87.6 | 22.4 |
| MRT_{0-inf} (h) | 71.8 | 104 | 87.9 | 22.7 |
| Tₗₐₛₜ (h) | 288 | 504 | 396 | 153 |
| AUC₀₋ₗₐₛₜ (h * μg/mL) | 30515 | 39434 | 34975 | 6307 |
| AUC_{0-inf} (h * μg/mL) | 30516 | 39469 | 34993 | 6331 |

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

### REFERENCES

[1] Alsaab H O, Sau S, Alzhrani R, et al. PD-1 and PD-L1 Checkpoint Signaling Inhibition for Cancer Immunotherapy: Mechanism, Combinations, and Clinical Outcome. Frontiers in Pharmacology 2017; 8: 561.
[2] Francisco L M, Sage P T, Sharpe A H. The PD-1 pathway in tolerance and autoimmunity. Immunological Reviews 2010; 236: 219-42.
[3] Gong, Jun, Chehrazi-Raffle, Alexander et al. Development of PD-1 and PD-L1 inhibitors as a form of cancer immunotherapy: a comprehensive review of registration trials and future considerations. Journal for Immunotherapy of Cancer 2018; 6: 8.
[4] Justin M. David et al. A novel bifunctional anti-PD-L1/TGF-β Trap fusion protein (M7824) efficiently reverts mesenchymalization of human lung cancer cells. Oncoimmunology. 2017; 6(10): e1349589.

1. A fusion protein, comprising an antibody or antigen-binding portion thereof that specifically binds to PD-L1 fused with a human transforming growth factor β receptor (TGFβR) or a portion thereof capable of binding to TGFβ, wherein the antibody or antigen-binding portion thereof comprises:
a heavy chain CDR1 (HCDR1) comprising the amino acid sequence of SEQ ID NO: 1; a HCDR2 comprising the amino acid sequence of SEQ ID NO: 2; a HCDR3 comprising the amino acid sequence of SEQ ID NO: 3; a light chain CDR1 (LCDR1) comprising the amino acid sequence of SEQ ID NO: 4; a LCDR2 comprising the amino acid sequence of SEQ ID NO: 5; and a LCDR3 comprising the amino acid sequence of SEQ ID NO: 6;
wherein the human TGFβR is TGFβRII.

2. (canceled)

3. The fusion protein of claim 1, wherein the human TGFβR or a portion thereof capable of binding to TGFβ comprises:
(A) an amino acid sequence which is at least 85%, at least 90%, or at least 95% identical to the amino acid sequence of the extra-cellular domain of the human TGFβRII; or
(B) a portion of the human TGFβRII which retains at least 75%, at least 80%, at least 85%, at least 90%, or at least 95% of the binding capacity to TGFβ.

4. The fusion protein of claim 1, wherein the TGFβR or a portion thereof comprises or consists of the amino acid sequence of SEQ ID NO: 9.

5. The fusion protein of claim 1, wherein the antibody or antigen-binding portion thereof comprises a heavy chain variable region (V_{H}) and a light chain variable region (V_{L}), wherein the V_{H} comprises:
(A) an amino acid sequence as set forth in SEQ ID NO: 7;
(B) an amino acid sequence which is at least 85%, at least 90%, or at least 95% identical to SEQ ID NO: 7; or
(C) an amino acid sequence with addition, deletion and/or substitution of one or more amino acids compared with SEQ ID NO: 7; and/or
the V_{L} comprises:
(A) an amino acid sequence as set forth in SEQ ID NO: 8;
(B) an amino acid sequence which is at least 85%, at least 90%, or at least 95% identical to SEQ ID NO: 8; or
(C) an amino acid sequence with addition, deletion and/or substitution of one or more amino acids compared with SEQ ID NO: 8.

6. The fusion protein of claim 1, wherein the antibody or antigen-binding portion thereof is a full antibody, ScFv, Fab, F(ab′)2, or Fv fragment.

7. The fusion protein of claim 1, wherein V_{H} region of the antibody or antigen-binding portion thereof is operably linked to a Fc region, optionally the Fc region is IgG1 isotype.

8-10. (canceled)

11. The fusion protein of claim 9, wherein the linker is a peptide linker comprising the amino acid sequence of (G4S)n, with n=2-4.

12. (canceled)

13. The fusion protein of claim 1, wherein the fusion protein comprises a heavy chain comprising the amino acid sequence of SEQ ID NO: 10 and a light chain comprising the amino acid sequence of SEQ ID NO: 11.

14. An isolated nucleic acid molecule, comprising a nucleic acid sequence encoding the fusion protein as defined in claim 1.

15. A vector comprising the nucleic acid molecule of claim 14.

16. A host cell comprising the nucleic acid molecule of claim 14 .

17. A pharmaceutical composition comprising the fusion protein as defined in claim 1 and a pharmaceutically acceptable carrier.

18. A method for producing the fusion protein as defined in claim 1, comprising the steps of:
expressing the fusion protein in a host cell comprising a vector(s) encoding the fusion protein; and
isolating the fusion protein from the host cell.

19. A method for modulating an anti-tumor immune response in a subject, comprising administering to the subject the fusion protein as defined in claim 1 to the subject.

20. (canceled)

21. A method for preventing or treating cancer in a subject, comprising administering an effective amount of the fusion protein as defined in claim 1 to the subject.

22. The method of claim 21, wherein the cancer is selected from colon cancer, lymphoma, lung cancer, liver cancer, cervical cancer, breast cancer, ovarian cancer, pancreatic cancer, melanoma, glioblastoma, prostate cancer, esophageal cancer, and gastric cancer.

23. The method of claim 21 or 22, wherein the cancer is lung cancer.

24-28. (canceled)

29. A kit for treating or diagnosing cancer, comprising a container comprising the fusion protein as defined in claim 1.

30. The fusion protein of claim 6, wherein the antibody or antigen-binding portion thereof is a full antibody, and the fully antibody is a humanized antibody or a human antibody.

31. The fusion protein of claim 30, wherein the Fc region is operably linked to the N terminal of the human TGFβR or a portion thereof, optionally via a linker.


SEQUENCE LISTING (11 sequences)

SEQ ID NO: 1 - protein, 10 aa, Artificial sequence
  HCDR1
GFSLTENSVS

SEQ ID NO: 2 - protein, 16 aa, Artificial sequence
  HCDR2
AVWSSGSTDYNSALKS

SEQ ID NO: 3 - protein, 13 aa, Artificial sequence
  HCDR3
STYSNDFYYYFDY

SEQ ID NO: 4 - protein, 11 aa, Artificial sequence
  LCDR1
SGSELPKRYAY

SEQ ID NO: 5 - protein, 7 aa, Artificial sequence
  LCDR2
KDSERPS

SEQ ID NO: 6 - protein, 11 aa, Artificial sequence
  LCDR3
SSTYGDRKLPI

SEQ ID NO: 7 - protein, 121 aa, Artificial sequence
  VH
QVQLQESGPGLVKPSETLSLTCTVSGFSLTENSVSWIRQPPGKGLEWIGAVWSSGSTDYNSALKSRVTISRDTSKNQFSLKLSSVTAADTAVYYCTRSTYSNDFYYYFDYWGQGTMVTVSS

SEQ ID NO: 8 - protein, 108 aa, Artificial sequence
  VL
SYELTQPPSVSVSPGQTASITCSGSELPKRYAYWYQQKPGQSIVRVIYKDSERPSGISERFSGSSSGNTATLTISGTQAMDEADYYCSSTYGDRKLPIFGGGTKLTVL

SEQ ID NO: 9 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - protein, 606 aa, Artificial sequence
  HC
QVQLQESGPGLVKPSETLSLTCTVSGFSLTENSVSWIRQPPGKGLEWIGAVWSSGSTDYNSALKSRVTISRDTSKNQFSLKLSSVTAADTAVYYCTRSTYSNDFYYYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 214 aa, Artificial sequence
  LC
SYELTQPPSVSVSPGQTASITCSGSELPKRYAYWYQQKPGQSIVRVIYKDSERPSGISERFSGSSSGNTATLTISGTQAMDEADYYCSSTYGDRKLPIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS